(19) United States
(12) Patent Application Publication
(10) Pub. No.: US 2024/0188862 A1
(43) Pub. Date: Jun. 13, 2024

(54) COMPRESSION DEVICE, METHOD, AND KIT FOR EXTRACTING BLOOD FROM A FINGER

(71) Applicant: THE KIT COMPANY, INC., New York, NY (US)

(72) Inventors: Philip FUNG, San Francisco, CA (US); Erik SALAZAR, Los Angeles, CA (US); Roberto SANABRIA, San Francisco, CA (US)

(21) Appl. No.: 18/554,628

(22) PCT Filed: Apr. 6, 2022
(86) PCT No.: PCT/US2022/023748

§ 371 Date: Oct. 9, 2023

Related U.S. Application Data
(60) Provisional application No. 63/171,965, filed on Apr. 7, 2021.

(51) Int. Cl. A61B 5/15

(52) U.S. Cl. CPC A61B 5/150068; A61B 5/150022; A61B 5/150259

(57) ABSTRACT

A compression device for extracting blood from a finger comprises a plunger slidably retained within a handle and a flexible strap with a first end attached to the plunger and a second end attached to the handle. The flexible strap forms a loop encircling the finger. Sliding of the handle towards the plunger applies pulling tension on the second end of the flexible strap causing reduction in its perimeter and leading to applying circumferential compression of the finger positioned inside. Periodic application of finger compression and relaxation urges blood to be extracted from the finger and collected in a scalable container.

### CROSS-REFERENCE TO RELATED APPLICATIONS

The present application is a 35 U.S.C. 371 national phase filing of PCT/US2022/023748, filed on Apr. 6, 2022, which claims the benefit of and priority to U.S. Provisional Application No. 63/171,965, filed on Apr. 7, 2021, and entitled “COMPRESSION DEVICE, METHOD, AND KIT FOR EXTRACTING BLOOD FROM A FINGER”, all of which are herein incorporated by reference in their entirety.

### FIELD OF THE INVENTION

The field of the invention is devices and methods for taking samples of blood. More specifically, the device contemplated herein is used to assist in extracting blood from a finger.

### BACKGROUND

The following description includes information that may be useful in understanding the present invention. It is not an admission that any of the information provided herein is prior art or relevant to the presently claimed invention, or that any publication specifically or implicitly referenced is prior art.

Collection of a blood sample from a finger is a frequent procedure performed for the purpose of a subsequent blood analysis. Frequently, but not exclusively, a blood sample from a finger is collected from diabetics in order to determine their glucose level. In other cases, blood collection is performed by a subject in order to collect a blood sample and send it via mail for further analysis. In some circumstances, more than just a small drop of blood is needed to be collected, such as at least 100 microliters or more. A single prick using a lancet may not produce enough blood to satisfy that minimum required volume. Manual finger compression or finger “milking” is not performed consistently and may cause blood damage or release of additional fluids into the blood sample, that may, in turn, cause an erroneous test result. There is a need to provide a compression device that applies uniform and limited pressure to the finger in order to assist in extracting blood therefrom.

The COVID-19 pandemic has raised the importance of providing virtual medical and diagnostic services at the home of the subject so as to avoid a visit to the doctor's office. One limitation of such an arrangement is the lack of objective laboratory data that can be typically obtained from an office-drawn blood sample. There is a need for providing a way for a subject to collect blood at home using a kit containing necessary supplies and materials and mail the collected blood sample for further processing and analysis by a laboratory.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

A variety of mechanical or motorized devices for compression of a finger are known in the art, ranging from clamps and tourniquets designed to stop bleeding in an event of trauma to blood extraction devices designed to collect a blood sample from a finger. Many of these devices employ mechanisms that have the capability of inadvertently applying excessive pressure on the finger and, as a result, may cause harm by pinching the skin and underlying tissues. The need, therefore, exists for compression devices that are both effective for blood extraction purposes, and yet have a limited ability to compress the finger so as to be safe in operation.

A number of compression devices described in the art are complex in manufacturing and therefore are not suitable for a single-use application. Reuse of these devices has a risk of pathogen transmission from one subject to another via contact with drops of blood. The need exists for a compression device with a limited number of inexpensive parts so as to facilitate low cost to allow discarding the device after a single use and reducing the risk of cross-contamination between subjects.

Finally, ease of operation is important to facilitate self-administration of the compression device by the subject, such as in circumstances of home use. Many prior art devices are either too complex to allow use by a person without special training or may cause an inadvertent injury if used incorrectly. The need exists therefore for a compression device that cannot be misused and can be operated safely by a person without prior training.

### SUMMARY OF THE INVENTION

The inventive subject matter provides compression devices, methods, and kits aimed at facilitating blood extraction from a human finger. In particular, the compression device for extracting blood from a finger comprises a slider assembly and a flexible strap. The slide assembly, in turn, comprises a plunger slidably retained within a handle. The flexible strap features a first end attached to a distal end of the plunger and a second end attached to a distal end of the handle. The term “distal”, in the context of this description, is used to denote a component further away from the handle in a direction from the operating end of the device and towards the finger compression end of the device.

As both ends of the flexible strap are attached to the plunger and the handle, the flexible strap itself forms a loop with a circumference, or a perimeter, selected to be sufficient for the loop to loosely encircle the finger positioned inside. Activation of the device entails sliding of the handle towards the plunger and away from the finger inside the flexible strap. This activation is envisioned to be done by a single hand, preferably the second hand of the subject during self-application of the compression device. The handle and the plunger are sized and shaped to be retained by the second hand and squeezed together during operation.

The sliding of the handle towards the plunger causes the second end of the flexible strap to be pulled along with the moving distal end of the handle towards the plunger, resulting in a decrease of the loop, thereby causing tension of the loop around the finger placed therein. This in turn caused the application of a circumferential pressure on the finger aimed at squeezing the finger and extracting blood therefrom, following piercing of the tip of the finger with a suitable piercing implement, such as a lancet.

Since the compression device does not have any latch or lock to retain the handle and the plunger together when the finger is compressed by the flexible strap, the simple act of releasing tension between the handle and the plunger leads to a release of finger compression.

An elastic pad is advantageously positioned at the distal end of the plunger to face the pad of the finger. The purpose of the elastic pad is to redistribute the circumferential pressure more evenly around the finger and make the process of finger compression more comfortable for the subject.

According to the methods of extracting blood using the compression device described herein, one or a series of such finger compressions are performed in rapid successions, such as over about a minute or two. As the blood is urged from the tip of the finger by repeated finger compressions, it can be collected in a suitable vial or another appropriate blood collection container. To facilitate spill-free blood collection, an optional funnel may be used to direct blood drops towards the opening of the collection container.

The compression device described herein allows extracting more than just a drop of blood over a series of periodic compressions of the finger. In various examples, at least 100 microliters of blood are collected into the vial, which is configured to be closed and sealed after the process is complete.

In addition to the compression device and a suitable blood collection container, other necessary devices and supplies are provided as part of the kit for blood collection. Such additional devices include a removable funnel for directing blood, a piercing lancet, cleaning and disinfecting supplies, and a mailing package with a suitable pre-addressed label to be used after the blood extraction process is complete.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is a perspective front view of the compression device

FIG. 2 is a side view of the same, and

FIG. 3 is a perspective back view of the same.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

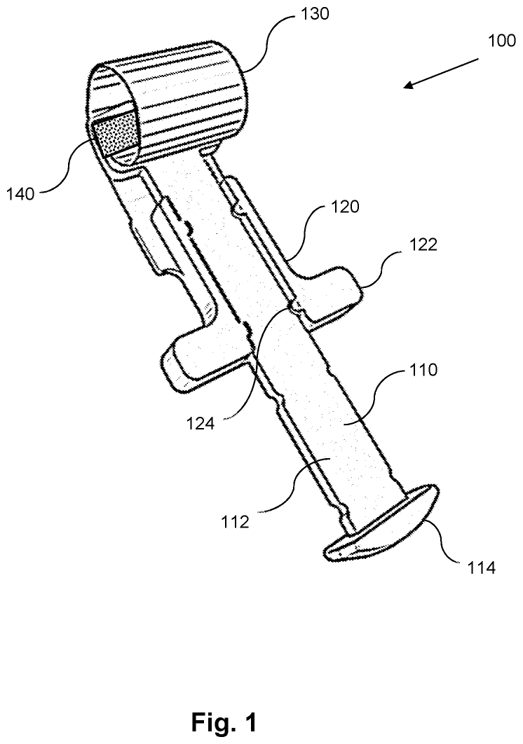

FIG. 1 shows an exemplary view of the compression device 100, which generally comprises a slider assembly comprising a plunger 110 slidably retained within a handle 120. The plunger 110 and the handle 120 are made from a rigid, preferably biocompatible or medical grade material, such as a suitable polymer, for example, polyethylene, polypropylene, polyurethane, acrylic, polyvinyl chloride, polyethersulfone, polytetrafluoroethylene, polyetherimide, polycarbonate, polysulfone, polyetheretherketone, or alike. A preferred material for the plunger and the handle is selected to be amenable to molding and other common manufacturing techniques and preferably low-cost.

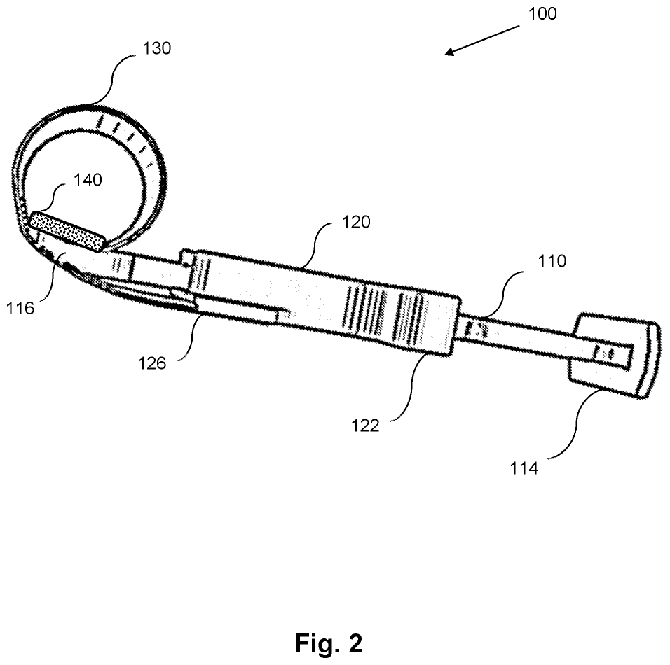

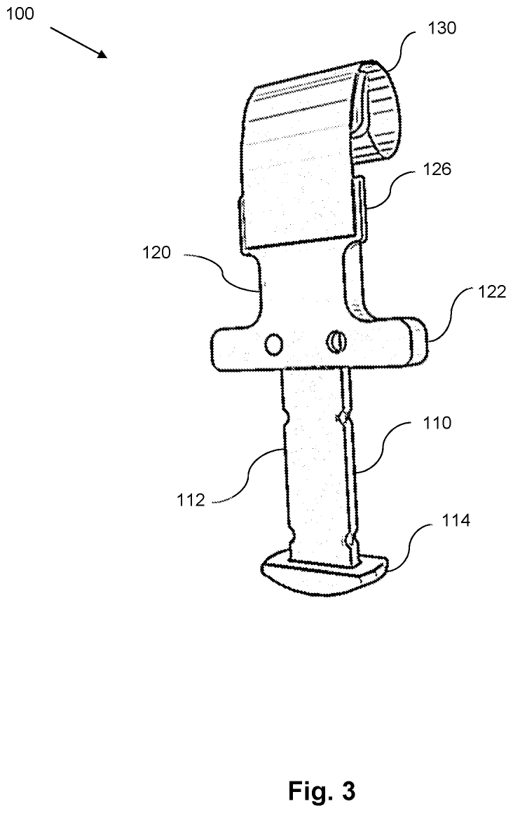

With respect to the remaining numerals in FIGS. 2 and 3, the same considerations for like components with like numerals of FIG. 1 apply.

The plunger 110 features a stem 112 having a distal end 116 and a proximal end equipped with a rest 114 configured to resting a thumb of a hand thereon during use. The stem 112 may be generally flat and have a rectangular shape as shown in FIG. 1 and FIG. 3. The length of the stem 112 is about 4 inches and the width is about 1 inch as it is configured for a one-handed operation described below. The distal end 116 of the plunger 110 may be made with a rounded edge seen best in FIG. 2. The purpose of the rounded edge is to facilitate the gliding of the flexible strap thereon with minimal friction and minimal deviation from the general shape of the loop as explained below in greater detail.

The handle 120 is made in a generally T-shape form with two prongs 122 extending on both sides thereof as seen in the figures. Two prongs 122 are suitably shaped for retention by fingers other than a thumb such that the entire slider assembly may be retailed by a single hand by holding the plunger rest and the two prongs 122 between the fingers of that hand. The compression device 100 thereby configured for operation using a single hand, which makes it advantageous during self-administration by the subject.

The extent of the two prongs 122 of the handle 120 and the size of the rest 114 are selected to allow for easy squeezing of the prongs 122 and the rest 114 together so as to slide the handle 120 towards the proximal end of the plunger 110. At the same time, operating the compression device 100 by one hand avoids applying excessive pressure as the force of such squeezing is naturally limited to avoid pinching or excessive compression of the hand.

Other hand engaging arrangements in addition, or in the alternative, to the two prongs 122 are also contemplated by the inventors. Such alternative designs include various openings for fingers, finger slots, finger grips, hand grip, etc.—all designed to allow a single hand operation of tightening the flexible strap 130 as described below in greater detail.

The handle 120 is made to slidably retain the plunger 110 therein. One advantageous configuration to accomplish such slidable retention is the use of several projections 124 positioned to define a sliding channel inside the handle 120 configured for the plunger stem 112 to reciprocally slide in.

A flexible strap 130 is provided as part of the compression device 100. The flexible strap is made from a non-elastic material, such as non-elastic fabric, for example cotton, polyester, silk, nylon or other polyamide fabrics, polyurethane fabrics (for example Lycra®; or spandex), or combinations thereof. A first end of the flexible strap is attached to the distal end of the plunger 116, for example by gluing, welding, or other known techniques. The second end of the flexible strap 130 is likewise attached to the distal end of the handle 120.

The free portion of the flexible strap 130 forms a free loop as best seen in FIG. 2. The length of the flexible strap is selected so as to form the loop with a perimeter suitable for encircling a finger placed inside. In examples, the length of the flexible strap is selected to be about 2 inches, or about 2.5 inches, or about 3 inches, or about 3.5 inches, or about 4 inches, or about 4.5 inches. A suitably shorter length of the flexible strap 130 is selected for a pediatric or a neonate version of the compression device 100, which are also contemplated by the inventors.

Explained from a different perspective, the loop formed by the free portion of the flexible strap is sized in its most extended state to accommodate a cylindrical object with a diameter ranging from 0 to about 1 inch, such as at least about 0.3 inches in diameter, or at least about 0.4 inches in diameter, or at least about 0.4 inches in diameter, or at least about 0.5 inches in diameter, or at least about 0.6 inches in diameter, or at least about 0.7 inches in diameter, or at least about 0.8 inches in diameter, or at least about 0.9 inches in diameter, or at least about 1 inch in diameter. Having a maximal size of the object that can fit inside the loop of the flexible strap as much as about 1 inch assures that one version of the compression device 100 can accommodate all sizes of human fingers, from the smallest children to the largest adults. Once the handle and the plunger are moved together, the perimeter of the free portion of the loop is reduced to zero or a small enough size to apply circumferential compression to a smallest human finger. That broad range assures that the compression device 100 is sized such that one size of the compression device fits all likely subjects. Having a single, universally usable device is a significant advantage of the compression device 100 as it simplifies logistics of using the device and the kit containing thereof in a variety of circumstances and for a broad range of subjects.

The width of the flexible strap is selected to cover a sufficiently long portion of the finger for compression and blood extraction. At the same time, the width of the flexible strap is selected to assure effective but not excessive finger compression when the device is manually operated as described in greater detail below. In various examples, the width of the flexible strap is selected to be about 0.5 inches, or about 0.6 inches, or about 0.7 inches, or about 0.8 inches, or about 0.9 inches, or about 1 inch, or about 1.1 inches, or about 1.2 inches, or about 1.3 inches, or about 1.4 inches, or about 1.5 inches, or about 1.6 inches, or about 1.7 inches. As mentioned above, for pediatric or neonate versions of the device, the width of the flexible strap 130 is selected to be appropriately lower.

The slider assembly is designed to allow sufficient length of the sliding motion of the handle 120 over the stem 112 of the plunger 110 in order to reduce the perimeter of the loop formed by the free portion of the flexible strap 130 from that allowing positioning of a finger inside thereof to that which causes compression of the finger. Given different sizes of various fingers due to anatomical differences between various subjects, the length of travel of the prongs 122 towards the plunger rest 114 is selected to be about or less than 3 inches, about or less than 2.75 inches, about or less than 2.5 inches, about or less than 2.25 inches, about or less than 2.0 inches, about or less than 1.75 inches, about or less than 1.5 inches, or about or less than 1.25 inches.

An elastic pad 140 is positioned on the distal end of the plunger 116 in such a way as to face the finger placed inside the flexible strap 130. As seen best in FIG. 2, the elastic pad 140 is positioned more distally than the point of attachment of the first end of the flexible strap 130 to the plunger 110. The purpose of the elastic pad is to redistribute the circumferential compression applied by the tightening of the flexible strap 130 and make the use of the compression device 100 more comfortable for the subject. The elastic pad 140 covers the entire width of the distal end 116 so that the finger rests thereon during use. The elastic pad 140 is made from a suitable medical-grade elastic material, such as rubber, elastic foam, neoprene, or another compressible or deformable material.

In other examples (not shown), a wider elastic pad 140 is placed with its ends surrounding a greater portion of the finger, in some cases as much as about ⅓, or ½, or even the entire finger periphery, so as to make the finger compression more comfortable.

In further examples of the compression device, the inventors have also contemplated the use of the elastic flexible strap. The elasticity of the strap is selected to further reduce the possibility of applying excessive compression of the finger as excessive squeezing of the device will simply result in elastic elongation of the flexible strap and prevent excessive compression of the finger.

According to the methods of blood extraction using the compression device described herein, the subject is instructed to pierce a tip of a finger on one hand with a lancet or another suitable piercing implement. The finger is then placed inside the loop of the flexible strap 130 such that the strap 130 is encircling and loosely covering a portion of the finger in the vicinity of the pierced tip. The finger is placed above the collection container, optionally over a funnel configured to direct blood from the finger towards the container opening. The compression device 100 is retained by another hand, such as for example with the thumb on the rest 114 and the prongs 122 retained between the index and the middle finger of the same hand. A squeezing motion is then manually applied, which causes a sliding motion of the handle 120 towards the rest 114 of the plunger 110. This in turn tangential pulling of the second end of the flexible strap 130 along with the handle 120, thereby decreasing the perimeter of the loop and tightening the flexible strap 130 around the finger positioned inside. Further squeezing of the compression device 100 by one hand causes circumferential compression of the finger, which urges blood to exit from the pierced tip of the finger and towards the collection container.

Release of the squeezing force relaxes the flexible strap 130 and restores normal blood circulation in the finger.

Repeated finger compression urges more blood to be extracted from the finger and collected in the container. In various contemplated examples, after at least one minutes or a few minutes of such alternating application of compression forces to the finger, at least 100 microliters, or at least 150 microliters, or at least 200 microliters, or at least 250 microliters, or at least 300 microliters, or at least 350 microliters, or at least 400 microliters, or at least 450 microliters, or at least 500 microliters of blood are extracted and collected in the suitable container, which is then closed and sealed.

In further examples of the method, the finger may be warmed up, massaged, or otherwise prepared before proceeding with the steps of blood extraction as described herein.

In addition to a slider assembly as described above, other mechanisms for one-handed tightening of the flexible strap are contemplated by the inventors. Examples of other suitable mechanisms include a variety of levers to amplify the pulling motion beyond the plunger-handle arrangement, mechanisms creating tension of the strap not by squeezing two parts of the mechanisms together but by separating one part from another, certain rotating mechanisms to cause tightening of the flexible strap by a twisting motion, etc.

A kit for collecting blood from a subject is envisioned by the inventors to include the compression device as described herein along with additional items and supplies needed for extracting and collecting blood. The kit includes a suitable piercing implement, cleaning and disinfecting supplies, a container for collecting blood, and an optional funnel configured to direct blood from the finger into the container, as well as appropriate packaging and mailing supplies and labels designed to provide the subject with the opportunity to complete the procedure and mail the extracted blood for further analysis at a laboratory. In further examples, the kit also includes a stand for retaining the collection vial in a vertical orientation.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In some embodiments, the numbers expressing quantities of components and their properties, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A compression device for extracting blood from a finger, the compression device comprising:
a slider assembly comprising a plunger slidably retained within a handle, and
a flexible strap with a first end attached to a distal end of the plunger and a second end attached to a distal end of the handle, the flexible strap forming a loop with a perimeter sufficient to encircle the finger when positioned inside thereof,
whereby manually sliding the handle towards the plunger causes tangential pulling of the second end of the flexible strap thereby decreasing the perimeter of the loop.

2. The compression device of claim 1, wherein the slider assembly is sized and configured to be manually activated.

3. The compression device of claim 2 configured for manual activation by one hand.

4. The compression device of claim 1, wherein the handle further comprises two prongs extending therefrom and sized to be retained between two fingers of a hand other than a thumb, the plunger is configured to be retained by the thumb of the same hand, whereby sliding of the handle towards the plunger causing tightening of the flexible strap and a decrease of the perimeter of the loop, thereby being capable of imparting circumferential compression of the finger positioned therein.

5. The compression device of claim 1, wherein cessation of sliding of the handle towards the plunder releases tangential tension and reduces circumferential compression of the finger encircled by the flexible strap.

6. The compression device of claim 1, wherein the distal end of the plunger has a width at least equal to the width of the flexible strap.

7. The compression device of claim 1, further comprising an elastic pad attached on the distal end of the plunger adjacent to and further distally from the first end of the flexible strap so as to face the finger encircled by the flexible strap, whereby sliding of the handle towards the plunger causes circumferential compression of both the finger resting on the elastic pad and the elastic pad itself.

8. The compression device of claim 7, wherein the elastic pad covers the entire width of the distal end of the plunger.

9. The compression device of claim 1, wherein the flexible strap is non-elastic.

10. (canceled)

11. The compression device as in claim 2, wherein the non-elastic fabric is made from a material selected from a group consisting of cotton, polyester, silk, nylon, polyurethane, polyamide, or a combination thereof.

12. The compression device of claim 1, wherein the flexible strap is elastic, whereby limiting the extent of circumferential compression of the finger encircled by the flexible strap.

13. The compression device of claim 1, wherein the width of the flexible strap is between 0.5 inches and 1.7 inches.

14. The compression device as in claim 13, wherein the width of the flexible strap is about 1 inch.

15. The compression device of claim 1, wherein the loop formed by the flexible strap is sized to be between 0.3 and 1 inch in diameter.

16. A method for extracting blood from a finger, the method comprising the following steps:
a. providing a manually activated compression device comprising a plunger slidably retained within a handle and a flexible strap with a first end attached to the plunger and a second end attached to the handle,
b. piercing a tip of the finger,
c. encircling the finger with a flexible strap of the compression device, and
d. operating the compression device to periodically tighten and relax the flexible strap so as to apply periodic circumferential compressions to the finger, whereby urging blood extraction therefrom.

17. The method of claim 16, wherein the compression device is configured for self-administration by a subject in need of extracting blood from their own finger.

18. The method of claim 16, wherein step (d) is repeated until at least 100 microliters of blood are collected from the finger.

19. The method of claim 16, wherein step (d) is repeated until at least 150 microliters of blood are collected from the finger.

20. The method of claim 16, wherein step (d) is repeated until at least 200 microliters of blood are collected from the finger.

21. The method of claim 16, wherein step (d) is repeated until at least 300 microliters of blood are collected from the finger.

22. The method of claim 16, wherein step (d) is repeated until at least 400 microliters of blood are collected from the finger.

23. The method of claim 16, wherein step (d) is repeated until at least 500 microliters of blood are collected from the finger.

24. The method of claim 16, wherein the compression device further comprises an elastic pad facing the finger upon encircling the finger with the flexible strap.

25. The method as in claim 24, wherein step (c) further comprising a step of positioning a pad of the finger over the elastic pad, whereby tightening of the flexible strap in step (d) applies distributed circumferential compression to the finger and avoids pinching thereof.

26. A kit for extracting blood from a finger via self-administration, the kit comprising:
a piercing implement,
the compression device of claim 1, and
a vial configured for collecting and storing of extracted blood.

27. A kit for extracting blood from a finger via self-administration, the kit comprising:
a piercing implement,
a manually-operated compression device, and
a vial configured for collecting and storing of extracted blood,
wherein the compression device in turn comprising a plunger slidably retained within a handle and a flexible strap with a first end attached to the plunger and a second end attached to the handle, and
wherein the compression device is configured to apply circumferential compression to the finger upon encircling thereof with the flexible strap and sliding of the handle and plunger together.
